(19) United States
(12) Patent Application Publication
(10) Pub. No.: US 2019/0091168 A1
(43) Pub. Date: Mar. 28, 2019

(54) FORMULATION FOR ENHANCED TRANSDERMAL ABSORPTION OF DRUG

(71) Applicants: UNIST(ULSAN NATIONAL INSTITUTE OF SCIENCE AND TECHNOLOGY), Ulsan (KR); SEOUL NATIONAL UNIVERSITY HOSPITAL, Seoul (KR)

(72) Inventors: Mi Hee Lim, Ulsan (KR); Sang Tae Kim, Seoul (KR); Ho Seong Han, Seoul (KR)

(73) Assignees: UNIST(ULSAN NATIONAL INSTITUTE OF SCIENCE AND TECHNOLOGY), Ulsan (KR); SEOUL NATIONAL UNIVERSITY HOSPITAL, Seoul (KR)

(21) Appl. No.: 15/885,085

(22) Filed: Jan. 31, 2018

(30) Foreign Application Priority Data
Sep. 26, 2017 (KR) 10-2017-0124525

(51) Int. Cl. A61K 9/70; A61K 47/14; A61K 47/12; A61K 47/60; A61K 31/4402; A61K 31/417; A61K 31/145; A61P 25/28; A61P 17/14

(52) U.S. Cl. CPC A61K 9/7069; A61K 47/14; A61K 47/12; A61K 47/60; A61P 17/14; A61K 31/417; A61K 31/145; A61P 25/28; A61K 31/4402

(57) ABSTRACT

Provided is a transdermal formulation including a transdermal absorption enhancer including an aliphatic hydrocarbon derivative and a pyrrolidone derivative; and a benzene diamine derivative for preventing or treating dementia, Alzheimer's disease, and hair loss.

### CROSS-REFERENCE TO RELATED APPLICATION

This application claims the benefit of Korean Patent Application No. 10-2017-0124525, filed on Sep. 26, 2017, in the Korean Intellectual Property Office, the disclosure of which is incorporated herein in its entirety by reference.

### BACKGROUND

### 1. Field

The present disclosure relates to a formulation for enhanced transdermal absorption of benzene diamine derivatives.

### 2. Description of the Related Art

Degenerative brain diseases are characterized by gradual occurrence of general impairment of mental or physical functions, which is caused by temporary or sustained damage to cranial nerves due to a variety of causes. There are more than 70 diseases reported as degenerative brain diseases. Of them, dementia, Alzheimer's dementia, Parkinson's disease, and frontotemporal degeneration are known as representative degenerative brain diseases.

Of them, dementia is one of diseases with high prevalence, and results from dysfunction of cerebral cortex, including memory, attention, language, and visual-spatial functioning, and thus patients with dementia suffer from many difficulties in daily or social lives.

Most of drugs that have been approved as dementia drugs by the FDA are directed to Alzheimer's dementia, and targets of the drugs are limited to acetylcholinesterase (e.g., Donepezil). However, inhibitors of this enzyme only function to inhibit acetylcholine reduction, leading to living an ordinary life, but they do not treat the underlying causes of dementia. In addition, there is a drug therapy using N-methyl-D-aspartate (NMDA) receptor antagonists. However, since this method is also based on ACh reduction in Alzheimer patients, it is not a fundamental method of treating Alzheimer's dementia. In other words, there are no drugs that ultimately restore the cause of dementia to a normal state.

In the case of patients with severe cognitive impairment among dementia patients, it is difficult to expect the patients voluntarily to take a medicine according to dosage and regimen of the medication guide without continuous care of caregivers. Accordingly, these patients require formulations for long-term continuous release of drugs.

Hair loss refers to abnormal loss of a large number of hairs due to a decrease in the number of hairs in the anagen phase and an increase in the number of hairs in the catagen or telogen phase during a growth cycle of hair. Compounds approved by the USFDA for the treatment or prevention of hair loss are finasteride and minoxidil. Finasteride is used as a prophylactic agent for hair loss, but its therapeutic effect on hair loss is poor. In addition, finasteride is known to affect male hormones. Because of teratogenic effects which may be caused by only skin contact, finasteride is contraindicated for use in women of childbearing age. Furthermore, finasteride is used only for oral administration. Minoxidil is used for external application. However, their mechanism of action on hair growth has not been clarified, and their therapeutic effects are also poor. Accordingly, there is a demand for a drug that may replace these drugs.

### SUMMARY

An aspect provides a transdermal formulation, the formulation including a transdermal absorption enhancer including an aliphatic hydrocarbon derivative and a pyrrolidone derivative; and a benzene diamine derivative.

Another aspect provides a transdermal patch having a drug-containing matrix layer which is formed on at least one side of a backing layer, the matrix layer including the transdermal absorption enhancer including the aliphatic hydrocarbon derivative and the pyrrolidone derivative; and the benzene diamine derivative.

### BRIEF DESCRIPTION OF THE DRAWINGS

These and/or other aspects will become apparent and more readily appreciated from the following description of the embodiments, taken in conjunction with the accompanying drawings in which:

FIG. 1 shows escape latencies which represent behavioral changes of dementia mouse models in a water maze test;

FIG. 2 shows changes in the skins of dementia and hair loss mouse models by Compound 2;

FIG. 3 shows SOX2 expression patterns after oral administration of Compound 2 to dementia and hair loss mouse models;

FIG. 4 shows SOX9 expression patterns after oral administration of Compound 2 to dementia and hair loss mouse models;

FIG. 5 shows SOX2 expression patterns after transdermal administration of Compound 2 to dementia and hair loss mouse models;

FIG. 6 shows SOX9 expression patterns after transdermal administration of Compound 2 to dementia and hair loss mouse models; and

FIG. 7 is a schematic illustration of a structure of a transdermal patch.

### DETAILED DESCRIPTION

An aspect provides a transdermal formulation, the formulation including a transdermal absorption enhancer including an aliphatic hydrocarbon derivative and a pyrrolidone derivative; and a benzene diamine derivative.

In a specific embodiment, the aliphatic hydrocarbon derivative may be isopropyl myristrate, corn oil PEG-8 ester, corn oil PEG-6 ester, lauryl alcohol, glycerol lauryl alcohol, oleyl alcohol, lauryl lactate, oleoyl macrogolglyceride, oleic acid, lauroyl macrogolglyceride, linoleoyl macrogolglyceride, glycerol monolaurate, glycerol monooleate, isopropyl myristrate, propylene monolaurate, propyleneglycol laurate, sorbitan monolaurate, sorbitan monostearate monooleate, sorbitan monooleate, propyleneglycol monolaurate, propylene monooleate, propyleneglycol monooleate, oleoyl macrogolglyceride, oleic acid, lauroyl macrogolglyceride, linoleoyl macrogolglyceride, propyleneglycol caprylate, propyleneglycol caprate, caprylic triglyceride, capric triglyceride, caprylic-capric triglyceride, propyleneglycol monocaprylate, polyoxyethylene monooleate, polyglyceryl diisostearate, sorbitan monostearate, or a combination thereof.

In a specific embodiment, the transdermal absorption enhancer may include glycerol monolaurate, glycerol monooleate, or a combination thereof.

In a specific embodiment, the transdermal absorption enhancer may include two or more aliphatic hydrocarbon derivatives. The two or more aliphatic hydrocarbon derivatives may include at least glycerol monolaurate and glycerol monooleate; glycerol monolaurate and sorbitan monolaurate; glycerol monooleate and sorbitan monolaurate; or glycerolmonolaurate, glycerol monooleate, and sorbitan monolaurate.

In a specific embodiment, the pyrrolidone derivative may be N-cyclohexyl-2-pyrrolidone, 1-butyl-3-dodecyl-2-pyrrolidone, 1,5-dimethyl-2-pyrrolidone, 1-ethyl-2-pyrrolidone, 1-hexyl-4-methyloxycarbonyl-2-pyrrolidone, 1-hexyl-2-pyrrolidone, 1-(2-hydroxyethyl)pyrrolidone, 3-hydroxy-N-methyl-2-pyrrolidone, lauryl pyrrolidone, 1-lauryl-4-methyloxycarbonyl-2-pyrrolidone, N-methyl-2-pyrrolidone, N-carprylyl-2-pyrrolidone, N-dodecyl-2-pyrrolidone, or a combination thereof.

In a specific embodiment, the transdermal absorption enhancer may include N-dodecyl-2-pyrrolidone.

In a specific embodiment, the benzene diamine derivative may be N¹-(pyridin-2-ylmethyl)benzene-1,4-diamine

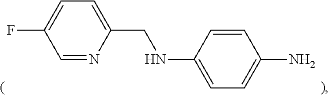 [SMILES: Nc1ccc(NCc2ccc(F)cn2)cc1]

N¹-((5-fluoropyridin-2-yl)methyl)benzene-1,4-diamine

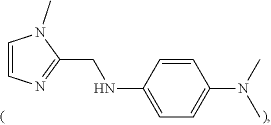 [SMILES: CN(C)c1ccc(NCc2nccn2C)cc1]

N¹,N¹-dimethyl-N⁴-((1-methyl-1H-imidazol-2-yl)methyl)benzene-1,4-diamine

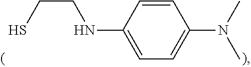

2-((4-(dimethylamino)phenyl)amino)ethane-1-thiol

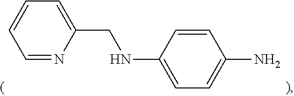 [SMILES: Nc1ccc(NCc2ccccn2)cc1]

or a combination thereof.

In a specific embodiment, the transdermal absorption enhancer may further include a citric acid derivative.

In a specific embodiment, the citric acid derivative may be acetyl triethyl citrate, diethyl citrate, tributyl citrate, triethyl citrate, acetyl tributyl citrate, or a combination thereof.

In a specific embodiment, the citric acid derivative may be triethyl citrate.

In a specific embodiment, the transdermal absorption enhancer may further include triacetin.

In a specific embodiment, the transdermal absorption enhancer may further include polyoxyethylene.

In a specific embodiment, a total content of the aliphatic hydrocarbon derivative and the pyrrolidone derivative in the transdermal absorption enhancer may be 15 w/w % or more. A total content of the aliphatic hydrocarbon derivative in the transdermal absorption enhancer may be 10 w/w % or more. In another specific embodiment, the total content of the aliphatic hydrocarbon derivative in the transdermal absorption enhancer may be 12.5 w/w % or more. A content of the pyrrolidone derivative in the transdermal absorption enhancer may be 2.5 w/w % or more. A content of triacetin in the transdermal absorption enhancer may be 5 w/w % or more. A content of the citric acid derivative in the transdermal absorption enhancer may be 5 w/w % or more. A content of the benzene diamine derivative in the transdermal absorption enhancer may be 1 w/w % or more.

The benzene diamine derivative included in the transdermal absorption enhancer may be in a form of being bound to a pharmaceutically acceptable salt.

The ‘pharmaceutically acceptable salt’ refers to a salt which has effective action relatively non-toxic and harmless to patients, and whose side effects do not degrade the beneficial efficacy of the compound in the composition of the present disclosure. The pharmaceutically acceptable salt refers to any organic or inorganic addition salt of the compound. The salt may use an inorganic acid and an organic acid as a free acid. The inorganic acid may be hydrochloric acid, bromic acid, nitric acid, sulfuric acid, perchloric acid, phosphoric acid, etc. The organic acid may be citric acid, acetic acid, lactic acid, maleic acid, fumaric acid, gluconic acid, methane sulfonic acid, gluconic acid, succinic acid, tartaric acid, galacturonic acid, embonic acid, glutamic acid, aspartic acid, oxalic acid, (D) or (L) malic acid, maleic acid, methane sulfonic acid, ethane sulfonic acid, 4-toluene sulfonic acid, salicylic acid, citric acid, benzoic acid, malonic acid, etc. In addition, these salts include alkali metal salts (sodium salts, potassium salts, etc.) and alkaline earth metal salts (calcium salts, magnesium salts, etc.). For example, the acid addition salt may include acetate, aspartate, benzoate, besylate, bicarbonate/carbonate, bisulfate/sulfate, borate, camsylate, citrate, edisilate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthalate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salt, etc., and among them, hydrochloride or trifluoroacetate may be used.

The transdermal formulation may be suitable for external application of the benzene diamine derivative. External preparations may be applied in a form of a patch, a band, an emulsion, an ointment, a pack, a gel, a cream, a lotion, a liquid, or a powder. As a cosmetic, a skin softener, a nutrient lotion, a massage cream, a nutrient cream, a moisturizing cream, a functional cream, a mist, a pack, a gel, or a skin adhesive-type formulation may be applied. Therefore, to be used for external application, ingredients commonly used in external preparations such as cosmetics or drugs, for example, an aqueous ingredient, an oily ingredient, a powdery ingredient, alcohols, a moisturizing agent, a thickener, an UV absorbing agent, a whitening agent, a preservative, an antioxidant, a surfactant, a flavoring agent, a colorant agent, several skin nutrition agents, etc. may be properly blended with the composition, as needed. The external preparations may be properly blended with a sequestering agent, such as disodium edetate, trisodium edetate, sodium citrate, sodium polyphosphate, sodium metaphosphate, gluconic acid, etc., a drug, such as caffeine, tannin, verapamil, licorice extract, glabridin, a hot water extract of fruit of carlin, various crude drugs, tocopherol acetate, glycyrrhizic acid, tranexamic acid, and their derivatives or salts, vitamin C, magnesium phosphate ascorbate, glucoside ascorbate, albutin, kojic acid, and sugars such as glucose, fructose, trehalose, etc.

It was confirmed that the benzene diamine derivatives of the present disclosure have an effect of inhibiting Aβ aggregation and an effect of alleviating dementia symptoms in dementia animal models. Aβ protein involved in dementia and Alzheimer's disease is well known, and high Aβ precipitation or aggregation is observed in Parkinson's disease, which has been reported to be closely associated with Parkinson's disease dementia commonly observed in Parkinson's disease (Mov Disord. November; 11(6) (1996): 647-53, etc.). It was also reported that excessive Aβ may promote hyperphosphorylation of tau protein, which is associated with formation of neurofibrillary tangles in frontotemporal degeneration (Curr Alzheimer Res. 2010 December; 7(8): 656-664). Therefore, in a specific embodiment, the transdermal formulation may be to prevent or treat degenerative brain diseases. The degenerative brain diseases may include dementia, Alzheimer's dementia, Parkinson's disease, or frontotemporal degeneration.

In a specific embodiment, the degenerative brain disease may be dementia or Alzheimer's dementia.

It was confirmed that transdermal administration of the benzene diamine derivative of the present disclosure increases SOX2 and SOX9 expressions and promotes hair growth in hair loss mouse models. Therefore, in a specific embodiment, the transdermal formulation may be to prevent or treat hair loss.

Another aspect provides a transdermal patch having a drug-containing matrix layer which is formed on at least one side of a backing layer, the matrix layer including the transdermal absorption enhancer including the aliphatic hydrocarbon derivative and the pyrrolidone derivative; and the benzene diamine derivative. The transdermal patch may be for transdermal administration of benzene diamine derivatives. In a specific embodiment, the transdermal patch may be in form of an adhesive bandage.

The transdermal patch may include a backing layer (100), a matrix layer (200), and a release layer (300), as shown in FIG. 7.

The backing layer (100) is to support the drug-containing matrix layer on at least one side thereof, and to fix a shape of the matrix layer, to prevent a hardening phenomenon due to drying after attachment, to block leakage of a drug to the outside, or to prevent entry of external contaminants into the matrix layer. As a material of the backing layer, a variety of plastic films such as polyurethane, polyester, polyolefin, polyvinyl chloride, polyvinylidene chloride, an ethylene/vinyl acetate copolymer, a pulp, a metal foil, a foam sheet, a woven fabric, a nonwoven fabric, a knitted fabric, a hollow fiber sheet using a hollow fiber yarn, etc., and a laminated complex film thereof, etc. may be used, but is not limited thereto.

The matrix layer (200) includes a drug, and the drug is an active ingredient for treating a primary indication. The drug may be a benzene diamine derivative, but is a concept including an auxiliary ingredient for enhancing the drug, for example, a nutrient, an enzyme, a complex active ingredient, etc. The matrix layer includes the benzene diamine derivative as an active ingredient and the transdermal absorption enhancer including an adhesive. The adhesive may be polyethylene vinyl acetate, styrene-isoprene-styrene, styrene-ethylene-propylene, styrene-butadiene-styrene, or a combination thereof. The adhesive may be included in an amount of 50 w/w % in the transdermal absorption enhancer. The matrix layer may be designed to include microneedles, thereby further enhancing absorption of the active ingredient.

The release layer (300) may be used in the same sense as a term such as a release paper, a release film, a release liner, etc., and the release layer may be selectively formed. The release layer plays a role in protecting the matrix layer during storage of the formulation, and it may be a portion to be detached and removed when used. The release layer may be prepared as a silicone resin- or fluoride resin-treated release paper, a metal foil, an aluminum film, a plastic film, a polyethylene film, a fluorocarbon diacrylate-coated film, etc.

When transdermal administration of a benzene diamine derivative may be performed by using a transdermal formulation including the benzene diamine derivative according to an aspect, compliance of patients may be greatly increased, long-term sustained release of the drug may be realized, and side effects of the drug may be minimized, as compared with oral formulations, injectable formulations, etc.

Another aspect provides a method for preventing, treating, or improving degenerative brain diseases or hair loss, comprising administrating any one formulation of the above-described transdermal formulation to a subject in need thereof. The subject may be a mammal, for example, a human, a cow, a horse, a pig, a dog, a sheep, a goat, or a cat, and the mammal may be a human. An administration dose of the formulation of the present disclosure effective for the human body may vary depending on age, body weight, and sex of a patient, administration mode, health conditions, and disease severity.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. Expressions such as “at least one of”, when preceding a list of elements, modify the entire list of elements and do not modify the individual elements of the list.

Hereinafter, the present invention will be described in more detail with reference to Examples. However, these Examples are for illustrative purposes only, and the scope of the present invention is not intended to be limited by these Examples.

### 1. Preparation Example 1: Synthesis of Benzene Diamine Derivatives

Benzene diamine derivatives were synthesized as follows.

### 1-1. Synthesis of N¹-(pyridin-2-ylmethyl)benzene-1,4-diamine (Compound 2)

### (1) Step 1

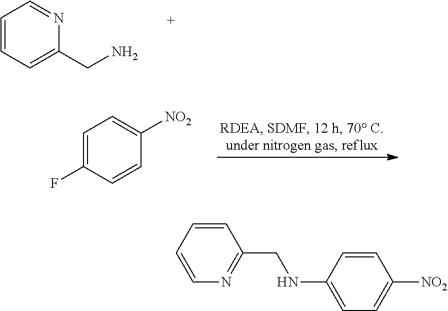 [SMILES: NCc1ccccn1 ; O=[N+]([O-])c1ccc(F)cc1 ; O=[N+]([O-])c1ccc(NCc2ccccn2)cc1]

Under a nitrogen atmosphere, 1-fluoro-4-nitrobenzene (231 μl, 2.2 mmol), N,N-diisopropylethylamine (836 μl, 4.8 mmol), and DMF (25 mL) were put in a well-dried flask equipped with a reflux condenser and a magnetic stirrer, and then 2-(aminoethyl)pyridine (247 μl, 2.4 mmol) was added at room temperature, and this mixture was heated at 70° C. 12 hours later, water (75 mL) was added to a resulting brown solution, and extracted with EtOAc (3×75 mL). An extracted organic solution was washed with water (2×75 mL) and brine (75 mL). Thereafter, MgSO₄ was added thereto, and filtration was performed, and then a filtrate was concentrated. This concentrated solution was purified by silica column chromatography (EtOAc in 25% to 100% n-hexane) to obtain a yellow solid compound (0.29 g, yield: 58%). [TLC conditions (EtOAc:n-hexane=50:50 (v/v)): Rf=0.25].

### (2) Step 2

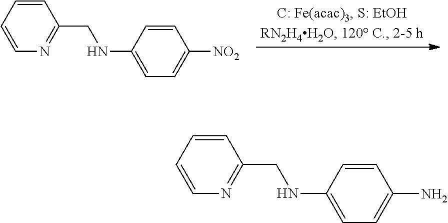 [SMILES: O=[N+]([O-])c1ccc(NCc2ccccn2)cc1 ; Nc1ccc(NCc2ccccn2)cc1]

The compound (0.52 g, 2.3 mmol) obtained in Step 1, tris(acetylacetonato)iron (III) (0.024 g, 3 mol %), and ethanol (20 mL) were put in a well-dried flask equipped with a reflux condenser and a magnetic stirrer, and then mixed with hydrazine hydrate (581 μl, 11 mmol). This mixture was heated under reflux for 2 hours at 120° C. 2 hours later, when the reactants remained, 4 equivalents of hydrazine hydrate was further added every hour. Resulting brown oil was concentrated and purified by silica column chromatography [EtOAc:Et₃N (99%:1%) in EtOAc (100%); TLC conditions: (EtOAc:Et₃N=99:1 (v/v)), Rf=0.20]. The purified compound was dissolved in a small amount of MeOH, and excess 5 M HCI was added thereto to produce a compound in the form of a salt. This product was concentrated under vacuum and then washed with Et₂O (3×5 mL). This resulting compound was dissolved in water (20 mL) to produce an aqueous layer, which was washed with Et₂O (3×20 mL), and concentrated. Water was removed under vacuum, and then recrystallization was performed by using MeOH and Et₂O. A product was a light yellow solid (0.45 g. yield: 85%).

### 1-2. Synthesis of N¹-((5-fluoropyridin-2-yl)methyl)benzene-1,4-diamine (Compound 3)

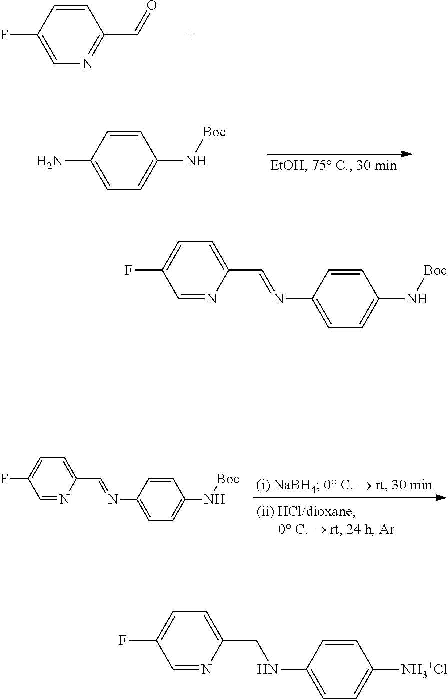

5-fluoropyridine-2-carboxaldehyde (20 mg, 0.152 mmol) was added to an ethanol solution (EtOH; 1.5 mL) containing N-(tert-butoxycarbonyl)-1,4-phenylenediamine (33.3 mg, 0.152 mmol). This reaction mixture was stirred at 75° C. for 24 hours. The solvent was concentrated under vacuum without other purification procedure to obtain a yellow solid product (50.4 mg, 0.152 mmol). Sodium borohydride (NaBH₄, 34.5 mg, 0.911 mmol) was added to methanol (2 mL, cooled to 0° C.) to obtain a solution of an imine product. This solution was stirred at 0° C. for 5 minutes. After 30 minutes at room temperature, this reaction was quenched with H₂O, and extracted with ethyl acetate (EtOAc, 3×). This mixed organic phase was washed with brine (lx) and dried over anhydrous magnesium sulfate (MgSO₄). This crude compound was purified by column chromatography (SiO₂, EtOAc: hexanes=1:6 to 1:2) to obtain a white solid product (33.9 mg, 0.107 mmol, 70.4%). An HCI/dioxane (4 mL, 4.0 M) solution was cooled at 0° C. under Ar (g) in a round-bottom flask equipped with a magnetic stirrer. A Boc-protected compound was added, and then the reactant was stirred at room temperature. 24 hours later, the solvent was concentrated under vacuum. Residues were washed with diethyl ether (Et₂O) and filtered and collected to obtain a yellow solid product (10.2 mg, 0.04 mmol, 37.6%). ¹H NMR [400 MHz, DMSO-d6, δ (ppm)]: 10.0 (3H, s), 8.63 (1H, d, J=3.2 Hz), 7.83 (1H, td, J=2.8 Hz, J=12.4), 7.55 (1H, m), 7.14 (2H, d, J=8.8 Hz), 6.77 (2H, d, J=8.8 Hz), 4.47 (6H, s). ¹³C NMR [100 MHz, DMSO-d6, δ (ppm)]: 160.1, 157.6, 154.7, 146.5, 136.4, 126.01, 124.2, 122.1, 114.7, 48.2. ESI-MS (m/z): [M+H]⁺ Calcd. for C₁₂H₁3FN₃⁺, 218.1; found, 218.0.

### 1-3. Synthesis of N¹,N¹-dimethyl-N⁴-((1-methyl-1H-imidazol-2-yl)methyl)benzene-1,4-diamine (Compound 4)

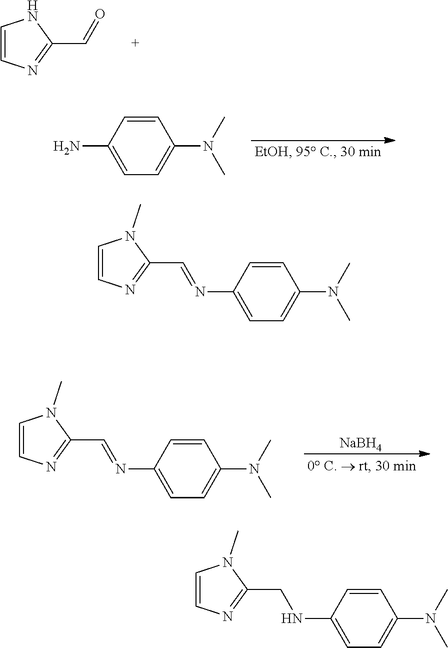

1H-imidazole-2-carbaldehyde (84.7 μl, 0.88 mmol) and N¹,N-dimethylbenzene-1,4-diamine (115 μl, 0.88 mmol) were put in a test tube containing EtOH (1.2 mL) with a magnetic stirrer. This reaction mixture was stirred at 9500 under N₂ (g) for 30 minutes. The solution was cooled to 000, and then NaBH₄ (222 mg, 5.87 mmol) dissolved in MeOH (3 mL, cooled to 0° C.) was directly added to the imine product solution. This reaction mixture was stirred at room temperature for 30 minutes. The reaction was quenched with H₂O, and extracted with Et₂O (3×). A product (brown solid; 89.1 mg, 0.39 mmol, 44%) was obtained from Et₂O and hexane by recrystallization. ¹H NMR [400 MHz, DMSO-d6, δ (ppm)]: 8.51 (1H, s), 7.36 (1H, s), 7.27 (2H, d, J=8.8 Hz), 7.10 (1H, s), 6.77 (2H, d, J=8.8 Hz), 4.05 (3H, s), 2.93 (6H, s). ¹³C NMR [100 MHz, DMSO-d6, δ (ppm)]: 146.2, 143.6, 141.3, 126.4, 122.1, 115.8, 114.1, 42.2, 41.3, 32.8. ESI-MS (m/z): [M+H]⁺ Calcd. for C₁₃H₁₉N₄⁺, 231.2; found, 231.3.

### 1-4. Synthesis of 2-((4-(dimethylamino)phenyl)amino)ethane-1-thiol (Compound 5)

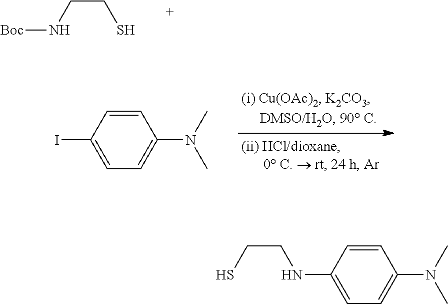

4-iodo-N,N-dimethylaniline (490 mg, 2.0 mmol), 2-(Boc-amino)ethanethiol (680 μl, 4.0 mmol), copper acetate dihydrate [Cu(OAc)₂.2H₂O; 40 mg, 0.2 mmol], and potassium carbonate (K₂CO₃; 1.1 g, 8.0 mmol) were put in a test tube with a magnetic stirrer containing DMSO/H₂O (3 mL/1 mL). After being flushed with Ar (g), the mixture was stirred in an oil bath preheated at 90° C. for 24 hours. This solution was cooled at room temperature, and then the reaction was quenched with EtOAc. The reaction solution was washed with H₂O (3×), and brine (lx). The organic layer was dried over MgSO₄, and concentrated under vacuum. A crude product was purified by column chromatography (SiO₂; EtOAc:hexane=1:10) to obtain a product (white solid; 210 mg, 0.71 mmol, 34%). tert-butyl 2-(4-(dimethylamino)phenylthio)ethyl carbamate (400 mg, 1.4 mmol) was added under Ar (g) at 0° C. to a HCI/dioxane (4.0 M, 10 mL) solution. This reaction mixture was stirred at room temperature for 24 hours. The solvent was removed under vacuum, and residues were washed with diethyl ether (Et₂O). A sticky compound was alkalized with 1 N NaOH (aq) and extracted with EtOAc (3×). The combined organic layer was washed with brine (lx), and dried over anhydrous MgSO₄, and concentrated under vacuum. A crude compound was purified by column chromatography (SiO₂; CH₂Cl₂:CH₃OH=7:1). A product (white powder; 50 mg, 0.25 mmol, 18%) was obtained by adding Et₂O to a yellow liquid product. ¹H NMR [400 MHz, CD₃OD, δ (ppm)]: 7.35 (2H, m), 6.72 (2H, d, J=8.8 Hz), 2.94 (10H, m), 1.90 (2H, s). ¹³C NMR [100 MHz, CD₃OD, δ (ppm)]: 152.2, 136.0, 119.2, 114.2, 40.6. 39.8, 35.5. HRMS (m/z): [M+H]+ Calcd. for C₁₀H₁₇N₂S+, 197.1107; found, 197.1103.

Compounds 2 to 5 thus obtained were freeze-dried to obtain dry powders. The obtained Compounds 2 to 5 were extracted simply with an aqueous solution and dried to obtain dry powders.

A variety of benzene diamine derivatives of the present disclosure may be prepared by a method well known to those skilled in the art, without being limited to the above-described Compounds 2 to 5.

### 2. Experimental Example 1: Preparation of Formulations for Transdermal Absorption and Comparison of Absorption Thereof

Formulations for transdermal absorption including the benzene diamine derivatives as active ingredients were prepared and their transdermal absorptions were compared.

### (1) Example 1

Toluene was added to 1% by weight of the dry powder of the benzene diamine derivative (Compound 2) prepared in Preparation Example 1, 50% by weight of EVA adhesive, 2.5% by weight of glycerol monolaurate, 10% by weight of glycerol monooleate, and 2.5% by weight of N-dodecyl-2-pyrrolidone (NP) such that a total weight of solids was 65% by weight, and completely dissolved to prepare an adhesive solution which is a transdermal absorption enhancer. Thereafter, a silicone-treated polyester release film was fixed on an applicator, and then the drug-containing adhesive solution was applied onto the polyester release film, and dried at 90° C. for 15 minutes at an air speed of 1500 rpm to prepare an adhesive layer sheet having a thickness of 100 μm. This adhesive layer sheet was combined with a polyester backing film, and cut into a predetermined size to prepare a transdermal formulation including the benzene diamine derivative of the present disclosure as an active ingredient.

### (2) Preparation of Examples 2 to 11

Formulations for transdermal absorption of Examples 2 to 11 were prepared by using ingredients of the following Table 1 in the same manner as in Example 1. In this regard, contents of respective ingredients are expressed as wt %.

TABLE 1

| | | Example | | | | | | | | | | |
|---|---|---|---|---|---|---|---|---|---|---|---|---|
| | | 1 | 2 | 3 | 4 | 5 | 6 | 7 | 8 | 9 | 10 | 11 |
| Ingredient (%) | Compound 2 | 1 | 1 | 1 | 1 | 1 | 1 | 1 | 1 | 1 | 1 | 1 |
| | EVA | 50 | 50 | 50 | 50 | 50 | 50 | 50 | 50 | 50 | 50 | 50 |
| | adhesive | | | | | | | | | | | |
| | GML | 2.5 | 2.5 | 2.5 | 2.5 | 2.5 | 2.5 | 2.5 | 2.5 | 2.5 | 2.5 | |
| | GMO | 10 | 10 | 10 | 10 | 10 | 10 | 10 | 10 | 10 | 10 | 10 |
| | SML | | 5 | | | | | | | | | |
| | NT | 2.5 | 2.5 | 2.5 | 2.5 | 2.5 | 2.5 | 2.5 | 2.5 | 2.5 | 2.5 | 2.5 |
| | CCS | | | 2.5 | | | | | | | | |
| | LL | | | | 2.5 | | | | | | | |
| | TA | | | | | 5 | 5 | 5 | 5 | 5 | 5 | 5 |
| | TC | | | | | | | | | | 5 | 5 |

EVA; polyethylene vinyl acetate adhesive,
GML; glycerol monolaurate,
GMO; glycerol monooleate,
SML; sorbitan monolaurate,
NP; N-dodecyl-2-pyrrolidone,
CCS; caprylic-capric triglyceride,
LL; lauryl lactate,
TA; triacetin,
TC; triethyl citrate

### (3) Preparation of Comparative Examples 1 to 15

Formulations for transdermal absorption of Comparative Examples 1 to 12 were prepared by using ingredients of the following Table 2 in the same manner as in Example 1. In this regard, contents of respective ingredients are expressed as wt %.

TABLE 2

| | | Comparative Example | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| Ingredient (%) | Compound 2 | 1 | 1 | 1 | 1 | 1 | 1 | 1 | 1 | 1 | 1 | 1 | 1 |
| | SIS | 50 | | | | | | | | | 50 | | |
| | Adhesive | | | | | | | | | | | | |
| | SEP | | 50 | | | | | | | | | 50 | |
| | Adhesive | | | | | | | | | | | | |
| | SBS | | | 50 | | | | | | | | | |
| | Adhesive | | | | | | | | | | | | |
| | Acrylic | | | | 50 | | | | | | | | |
| | Adhesive | | | | | | | | | | | | |
| | EVA | | | | | 50 | 50 | 50 | 50 | 50 | 50 | 50 | 50 |
| | Adhesive | | | | | | | | | | | | |
| | NP | 2.5 | 2.5 | 2.5 | 2.5 | | | | | | | | |
| | SML | | | | | 5 | | | | | | | |
| | IPM | | | | | | 5 | | | | | | |
| | PMO | | | | | | | 5 | | | | | |
| | PGML | | | | | | | | 5 | | | | |
| | LL | | | | | | | | | 5 | | | |
| | TA | | | | | | | | | | 5 | | |
| | TC | | | | | | | | | | | 5 | |
| | PGL | | | | | | | | | | | | 5 |

SLS adhesive; styrene-isoprene-styrene adhesive,
SEP adhesive; styrene-ethylene-propylene,
SBS adhesive; styrene-butadiene-styrene adhesive,
EVA adhesive; polyethylene vinyl acetate adhesive,
NP; N-dodecyl-2-pyrrolidone,
SML; sorbitan monolaurate,
IPM; isopropyl myristrate,
PMO; polyoxyethylene,
PGML; propyleneglycol monolaurate,
LL; lauryl lactate,
TA; triacetin,
TC; triethyl citrate,
PGL; propyleneglycol monolaurate

### (4) Drug Permeation Test

A drug permeation test was performed for the formulations of Examples 1 to 11 and Comparative Examples 1 to 12.

In detail, Franz cell which is a diffusion cell for drug permeation testing was filled with a phosphate buffer solution at pH 7.4 which was supplemented with 0.5% by weight of sodium azide to prevent skin putrefaction. This aqueous solution was maintained at a temperature of 32±0.500, and a frozen cadaveric scalp (PEA020, BIOPREDIC, France) was used as a skin model. The formulations of Examples 1 to 11 and Comparative Examples 1 to 12 were cut to the size of the scalp tissue. 2 days later, each 200 μl of the aqueous solutions was collected from the Franz cell, and the drug permeated into the scalp tissue was weighed by liquid chromatography. Average scalp permeation rates of the drugs at 2 days are shown in Tables 3 and 4 below.

TABLE 3

| | Skin permeation (μG/Cm²/hr) |
|---|---|
| Example 1 | 14.34 ± 0.45 |
| Example 2 | 14.32 ± 0.67 |
| Example 3 | 14.90 ± 1.21 |
| Example 4 | 15.78 ± 1.21 |
| Example 5 | 16.23 ± 1.34 |
| Example 6 | 17.45 ± 1.34 |
| Example 7 | 16.23 ± 1.78 |
| Example 8 | 18.23 ± 1.6 |
| Example 9 | 16.34 ± 2.1 |
| Example 10 | 23.12 ± 1.2 |
| Example 11 | 23.31 ± 1.1 |

TABLE 4

| | Skin permeation (μG/Cm²/hr) |
|---|---|
| Comparative Example 1 | 1.14 ± 0.25 |
| Comparative Example 2 | 1.32 ± 0.11 |
| Comparative Example 3 | 1.10 ± 0.41 |
| Comparative Example 4 | 1.28 ± 0.1 |
| Comparative Example 5 | 1.3 ± 0.12 |
| Comparative Example 6 | 1.23 ± 0.2 |
| Comparative Example 7 | 1.33 ± 0.13 |
| Comparative Example 8 | 1.43 ± 0.25 |
| Comparative Example 9 | 2.75 ± 0.1 |
| Comparative Example 10 | 3.03 ± 0.2 |
| Comparative Example 11 | 3.12 ± 0.5 |
| Comparative Example 12 | 1.34 ± 0.1 |

As a result, Examples 1 to 11 prepared by a combination of the allopathic derivative and the pyrrolidone derivative showed remarkably high transdermal permeation, as compared with Comparative Examples 1 to 12. As Examples 1 to 9 were compared with Examples 10 and 11, Examples 10 and 11 prepared by further adding triacetin and a citric acid derivative (triethyl citrate) showed remarkably high transdermal permeation.

### 3. Experimental Example 2: Examination of Effects of Benzene Diamine Derivatives by Using Animal Models

Therapeutic effects of the benzene diamine derivatives on diseases or symptoms were examined by using animal models. For this experiment, dementia animal models were introduced, and these dementia animal models are characterized by appearance of severe hair loss.

### (1) Experimental Example 2-1: Preparation of Animal Model

APP/PS/Tau was purchased as a dementia mouse model from Jackson lab (BAR HARBOR, USA), and APP/Tau female and APP/PSen1de9 purchased from Taconic (Hudson, USA) were cross-mated for 6 generations, and 4-month-old mouse models were obtained from F1.

### (2) Experimental Example 2-2: Examination of Therapeutic Effects on Dementia of Animal Model

In order to examine therapeutic effects of the benzene diamine derivative of the present disclosure on dementia, enhancement of spatial cognition and memory in dementia mouse models were investigated. For this investigation, a Morris water maze (Harvard Apparatus, USA) test was performed.

In detail, a water maze composed of a stainless steel pool with a diameter of 150 cm×a height of 60 cm and an escape platform (10 cm in diameter, 30 cm in height) was filled with water at 222° C., and the height was 2 cm above the platform such that when a mouse sat on the escape platform, the body was allowed to come out of the water. Since the mouse searches for the escape platform using a marker around the water tank on the surface of the water tank, the marker was kept constant during the experiment so that the environment did not change. Escape latency was measured as the time taken for the experimental animal to go to the escape platform when the experimental animal went to the escape platform and stayed thereon for more than 20 seconds. This test was performed three times a day, and a mean value was determined as mean escape latency. The escape latency was recorded on the computer program (Smart Video Tracking System V3.0, PanLab SL, Barcelona, Spain) by installing a camera on the ceiling above the water tank. The experiment was performed three times a day for 6 days, and at this time, the possibility of accidentally visiting the escape platform was minimized by sequentially changing the positions of the experimental animals in the water tank. In the experiment, a normal mouse, a control dementia mouse administered with a vehicle (PBS) and an experimental dementia mouse administered with 100 μg/kg of N¹-(pyridin-2-ylmethyl)benzene-1,4-diamine (Compound 2) twice a week for 2 months were used after completing administration.

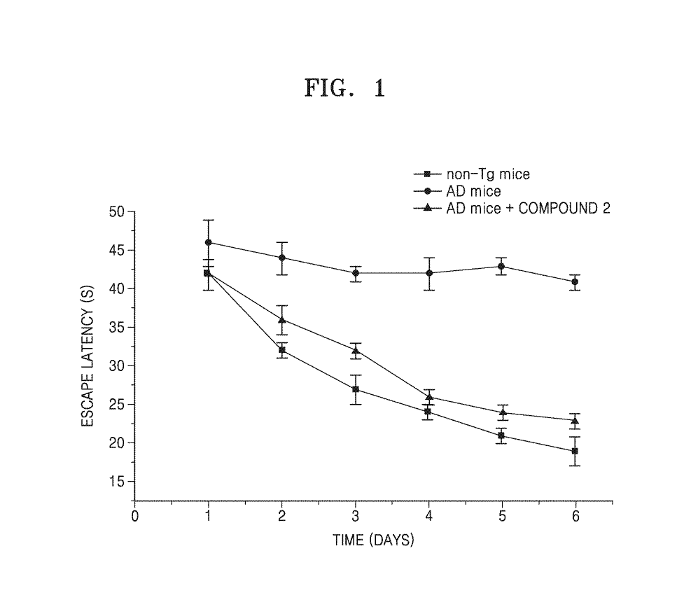

As a result, as compared with the dementia control group, the Compound 2-treated group showed significantly short mean escape latency at 3 days, which was a similar level to that of the normal group (non-Tg mice), as shown in FIG. 1. The control group (AD mice) and the experimental group (AD mice+Compound 2) showed the mean escape latency of 42±1 sec and 26±1 sec at 4 days, respectively, and the difference was 16 sec. The dementia group and the Compound 2-treated group showed the mean escape latency of 43±1 sec and 24±1 sec at 5 days, respectively, and the difference was 19 sec, indicating statistically significant recovery of spatial cognition and memory. In particular, the normal group and the Compound 2-treated group showed a stable reduction graph after 4 days, clearly indicating long-term memory formation during the experimental period.

### (2) Experimental Example 2-3: Therapeutic Effects on Hair Loss of Animal Model

In order to examine therapeutic effects of the benzene diamine derivative of the present disclosure on hair loss, the benzene diamine derivative was orally administered to hair loss-induced mouse models, and changes in appearance were examined. The animal model of Experimental Example 2-1 was characterized by appearance of hair loss as well as dementia symptoms. Therefore, the animal model of Experimental Example 2-1 was used as a hair loss mouse model.

The hair loss mouse models were divided into groups of 6 mice, and among the hair loss mouse models, the control group were orally administered with a phosphate buffer vehicle (PBS) for 2 months, and the experimental group was orally administered with 100 μl of N1-(pyridin-2-ylmethyl)benzene-1,4-diamine (Compound 2) at a concentration of 100 μg/Kg twice a week for 2 months. Thereafter, changes in their appearance were compared.

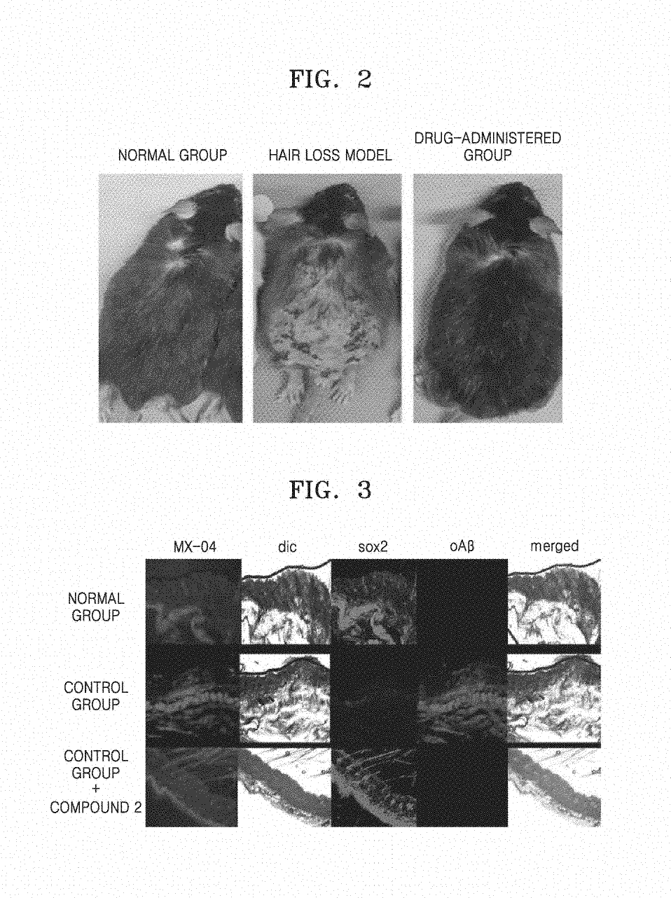

As a result, the mice administered with the control group showed bleeding and hair loss in the skin, whereas the experimental group administered with the benzene diamine derivative showed remarkable improvement in hair loss, similar to the normal group (FIG. 2).

### 4. Experimental Example 3: Histological Observation of Benzene Diamine Derivative

Changes in the animal models by the benzene diamine derivative were histologically examined.

In general, stratum corneum existing in the epidermis of the skin regularly slough off, and therefore, in order to make up stratum corneum, SOX-2 around the endothelial-dermal layer is a transcription factor essential for maintaining self-renewal or pluripotency of undifferentiated embryonic stem cells. SOX-2 is known to be critical for regulation of progenitor cells in hair growth (Dev Cell. 2012 Nov. 13; 23(5): 981-994). Dermal papilla cells (DP cells) are known to play a key role in inducing growth of hair follicles, and therefore, SOX-2 is an important contributor to normal DP function (Clavel et al., 2012). Further, growth of hair follicles is stimulated by stem cells in the anagen cycle, and during development of hair follicles, formation of hair follicle stem cells occurs, which requires the transcription factor, SOX-9. Therefore, SOX-9 plays a very important role in maintaining adult hair follicle stem cells.

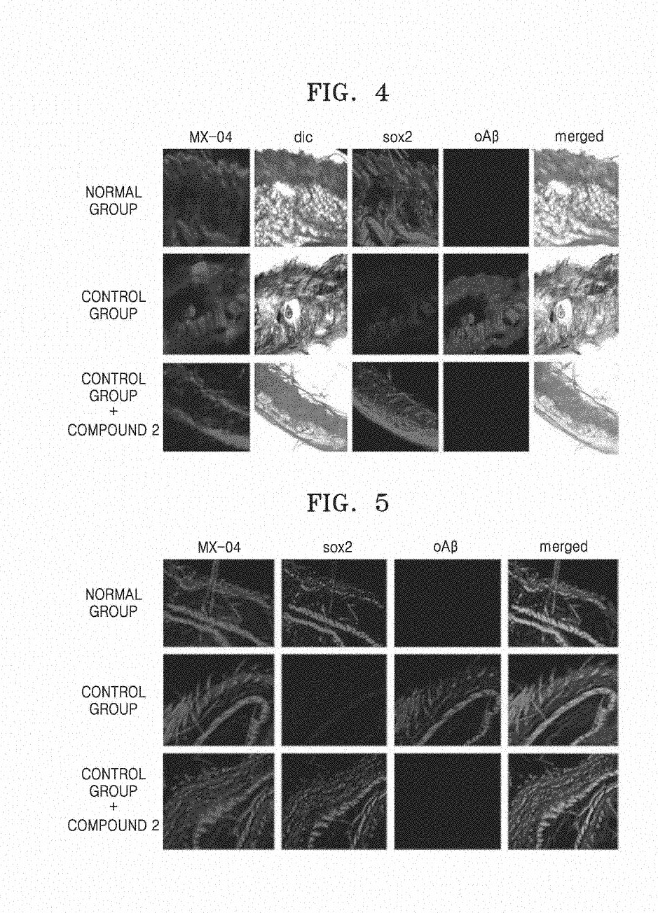

Accordingly, the present inventors examined whether expressions of hair growth stimulating factors, SOX-2 and SOX-9 were increased in hair loss animal models by the benzene diamine derivatives of the present disclosure. In order to examine SOX-2 and SOX-9 expressions inside skin tissues, APP/PS/Tau mouse which is a hair loss-induced mouse model was selected as a control group and C57BL6 mouse was selected as a normal group, as in Example 5. An experimental group was prepared by orally administering the control group with the benzene diamine derivative at a concentration of 100 μg/Kg/0.1 ml using a zonde twice a week. 2 months later, incision of the skin was performed under local anesthesia, and the skin tissues were fixed in 4% paraformaldehyde solution for 24 hours, and fixed in a plastic mold with O.C.T. Compound (SAKURA, USA) for cryostat sectioning. Thereafter, the tissues were frozen in a freezer for 5 hours, and sectioned at a thickness of 5 μm by using a freezing microtome (LEICA, JAPAN), and SOX-2/QD525 which is a conjugate of an antibody against Sox-2 and a fluorescent dye, SOX-9/QD525 against Sox-9, and m/oAβ-cy5.5 antibody solution against skin oligomer Aβ were used in a 24-well plate to induce an antigen-antibody reaction. Thereafter, the plate was put on a slide glass and covered with a cover slide, and stored in a dark acrylic box, and fluorescence imaging was observed under a confocal microscope (LSM710, Carl Zeiss, Germany) by 405/488/565 nm laser. As a result, SOX-2 expression was observed in the skin of the normal group, but SOX-2 was not expressed in the control group. Remarkable expression of oligomer Aβ was observed, as compared with the normal group. However, in the hair loss model administered with Compound 2, SOX-2 was remarkably expressed close to the normal group (FIG. 3). Similarly, in the control group, SOX-9 was rarely expressed, but monomer/oligomer Aβ was expressed. In contrast, in the hair loss model APP/PS/Tau administered with Compound 2, SOX-9 expression was remarkably increased, similar to the normal group, but monomer/oligomer Aβ expression was decreased. In all the three groups, MXO4 which is a blue fluorescent dye detecting plaques in the blood vessel was strongly observed (FIG. 4), suggesting that Compound 2 inhibits oligomer Aβ production rather than removes plaques.

### 5. Experimental Example 4: Examination of Effect of Transdermal Formulation by Using Animal Model

It was examined by using the dementia and hair loss animal model of Experimental Example 2-1 whether transdermal absorption of the benzene diamine derivative actually occurred to exhibit therapeutic effects on diseases and symptoms.

### (1) Preparation of Formulation for Transdermal Administration

For preparation of formulations for transdermal administration, toluene was added to 1% by weight of N¹-(pyridin-2-ylmethyl)benzene-1,4-diamine (Compound 2), 50% by weight of a polyethylene vinyl acetate adhesive, 10% by weight of glycerol monooleate, 5% by weight of triacetin, and 5% by weight of triethyl citrate and completely dissolved such that a total weight of solids was 65%. The drug-containing adhesive solution was coated on a silicone-treated polyester release film fixed on an applicator, and dried at 90° C. for 15 minutes at an air speed of 1500 rpm to prepare a sheet of an adhesive layer having a thickness of 100 μm, which was then bound to a polyester backing film. The resulting product was cut into a predetermined size to prepare a transdermal formulation.

### (2) Transdermal Administration

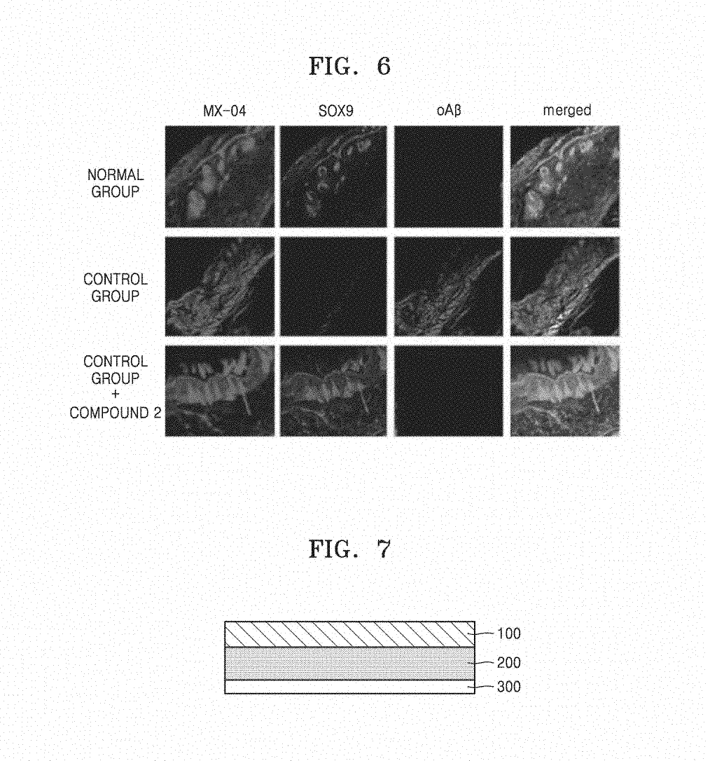

The transdermal formulation was attached to the back skin of the dementia and hair loss animal model APP/PS/Tau, and replaced every 2 weeks for 2 months. Incision of the skin of the animal model was performed under local anesthesia, and the skin tissues were fixed in 4% paraformaldehyde solution for 24 hours, and fixed in a plastic mold with O.C.T. Compound (SAKURA, USA) for cryostat sectioning. Thereafter, the tissues were frozen in a freezer for 5 hours, and sectioned at a thickness of 5 μm by using a freezing microtome (LEICA, JAPAN). Then, SOX-2/QD525 which is a conjugate of an antibody against Sox-2 and a fluorescent dye, SOX-9/QD525 against Sox-9, and m/oAβ-cy5.5 antibody solution against skin oligomer Aβ were used in a 24-well plate to induce an antigen-antibody reaction. Thereafter, the plate was put on a slide glass and covered with a cover slide, and stored in a dark acrylic box, and fluorescence imaging was observed under a confocal microscope (LSM710, Carl Zeiss, Germany) by 405/488/565 nm laser. SOX-2 expression was observed in the skin of the normal group, whereas SOX-2 was not observed in the control group. Remarkable expression of oligomer Aβ was observed in the control group, as compared with the normal group. However, in the hair loss model administered with the transdermal formulation including Compound 2, SOX-2 was remarkably expressed, close to the normal group (FIG. 5). Similarly, in the control group, SOX-9 was rarely expressed, but oligomer Aβ was expressed. In contrast, when the transdermal formulation including Compound 2 was administered, SOX-9 expression was remarkably increased, similar to the normal group, but oligomer Aβ expression was decreased. In all the three groups, MXO4 which is a blue fluorescent dye detecting plaques in the blood vessel was strongly observed (FIG. 6), suggesting that Compound 2 inhibits oligomer Aβ production rather than removes plaques. Accordingly, it was demonstrated that Compound 2 was absorbed into the skin tissue to increase SOX2 and SOX9 and to decrease monomer Aβ and oligomer Aβ, and therefore, it is easily predicted that the therapeutic effect on dementia may be obtained by transdermal administration as well as by oral administration.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

1. A transdermal formulation, comprising a transdermal absorption enhancer comprising an aliphatic hydrocarbon derivative, a pyrrolidone derivative, triacetin and a citric acid derivative; and a benzene diamine derivative.

2. The transdermal formulation of claim 1, wherein the benzene diamine derivative is N¹-(pyridin-2-ylmethyl)benzene-1,4-diamine, N¹-((5-fluoropyridin-2-yl)methyl)benzene-1,4-diamine, N¹,N¹-dimethyl-N⁴-((1-methyl-1H-imidazol-2-yl)methyl)benzene-1,4-diamine, 2-((4-dimethylamino)phenyl)amino)ethane-1-thiol, or a combination thereof.

3. (canceled)

4. (canceled)

5. The transdermal formulation of claim 1, wherein the transdermal absorption enhancer further comprises polyoxyethylene.

6. The transdermal formulation of claim 1, comprising two or more aliphatic hydrocarbon derivatives.

7. The transdermal formulation of claim 1, wherein the aliphatic hydrocarbon derivative is isopropyl myristrate, corn oil PEG-8 ester, corn oil PEG-6 ester, lauryl alcohol, glycerol lauryl alcohol, oleyl alcohol, lauryl lactate, oleoyl macrogolglyceride, oleic acid, lauroyl macrogolglyceride, linoleoyl macrogolglyceride, glycerol monolaurate, glycerol monooleate, isopropyl myristrate, propylene monolaurate, propyleneglycol laurate, sorbitan monolaurate, sorbitan monostearate monooleate, sorbitan monooleate, propyleneglycol monolaurate, propylene monooleate, propyleneglycol monooleate, oleoyl macrogolglyceride, oleic acid, lauroyl macrogolglyceride, linoleoyl macrogolglyceride, propyleneglycol caprylate, propyleneglycol caprate, caprylic triglyceride, capric triglyceride, caprylic-capric triglyceride, propyleneglycol monocaprylate, polyoxyethylene monooleate, polyglyceryl diisostearate, sorbitan monostearate, or a combination thereof.

8. The transdermal formulation of claim 1, wherein the aliphatic hydrocarbon derivative is glycerol monolaurate, glycerol monooleate, or a combination thereof.

9. The transdermal formulation of claim 3, wherein the citric acid derivative is acetyl triethyl citrate, diethyl citrate, tributyl citrate, triethyl citrate, acetyl tributyl citrate, or a combination thereof.

10. The transdermal formulation of claim 3, wherein the citric acid derivative is triethyl citrate.

11. The transdermal formulation of claim 1, wherein the pyrrolidone derivative is N-cyclohexyl-2-pyrrolidone, 1-butyl-3-dodecyl-2-pyrrolidone, 1,5-dimethyl-2-pyrrolidone, 1-ethyl-2-pyrrolidone, 1-hexyl-4-methyloxycarbonyl-2-pyrrolidone, 1-hexyl-2-pyrrolidone, 1-(2-hydroxyethyl)pyrrolidone, 3-hydroxy-N-methyl-2-pyrrolidone, lauryl pyrrolidone, 1-lauryl-4-methyloxycarbonyl-2-pyrrolidone, N-methyl-2-pyrrolidone, N-caprylyl-2-pyrrolidone, N-dodecyl-2-pyrrolidone, or a combination thereof.

12. The transdermal formulation of claim 1, wherein the pyrrolidone derivative is N-dodecyl-2-pyrrolidone.

13. The transdermal formulation of claim 1, wherein a total content of the aliphatic hydrocarbon derivative and the pyrrolidone derivative in the transdermal absorption enhancer is 15 w/w % or more.

14. The transdermal formulation of claim 1, wherein the transdermal formulation is to prevent or treat dementia or Alzheimer's dementia.

15. (canceled)

16. The transdermal formulation of claim 1, wherein the transdermal formulation is to prevent or treat hair loss.

17. A transdermal patch comprising a drug-containing matrix layer which is formed on at least one side of a backing layer, wherein the matrix layer comprises a transdermal absorption enhancer comprising an aliphatic hydrocarbon derivative and a pyrrolidone derivative; and a benzene diamine derivative.

18. A method for preventing, treating, or improving dementia, Alzheimer's dementia or hair loss, comprising administrating the transdermal formulation of claim 1 to a subject in need thereof.

19. (canceled)
